(19) United States
(12) Patent Application Publication
(10) Pub. No.: US 2014/0275636 A1
(43) Pub. Date: Sep. 18, 2014

(54) Catalysts For Forming Diethyl Ether

(71) Applicant: Celanese International Corporation, Irving, TX (US)

(72) Inventors: Cheng Zhang, Houston, TX (US); Victor J. Johnston, Houston, TX (US)

(73) Assignee: Celanese International Corporation, Irving, TX (US)

(21) Appl. No.: 14/212,420

(22) Filed: Mar. 14, 2014

Related U.S. Application Data
(60) Provisional application No. 61/788,028, filed on Mar. 15, 2013.

(51) Int. Cl. C07C 41/09

(52) U.S. Cl. CPC C07C 41/09
USPC 568/698

(57) ABSTRACT

A process is disclosed for producing of diethyl ether by dehydrating ethanol in the presence of an inventive catalyst. A preferred catalyst achieves high conversions of ethanol and high selectivites to diethyl ether. The diethyl ether may be used as a solvent, fuel additive, or fuel.

### CROSS REFERENCE TO RELATED APPLICATIONS

The present invention claims priority to U.S. Provisional No. 61/788,028, filed Mar. 15, 2013, the entire contents and disclosure of which are hereby incorporated by reference.

### FIELD OF THE INVENTION

The present invention relates generally to processes for preparing diethyl ether from ethanol and to inventive dehydrating catalysts for use in such processes, the catalysts having high selectivities for diethyl ether and preferably having low selectivity to ethylene. The diethyl ether may be used in solvents, fuel additives and fuels.

### BACKGROUND OF THE INVENTION

Diethyl ether finds various uses as a solvent, anesthesia, fuel additive and fuel. Currently diethyl ether is produced as a byproduct of the dehydration of ethanol to make ethylene using sulfuric acid, as described in U.S. Pat. Nos. 7,585,339, 5,227,141 and 3,847,756. Other processes, such as those described in U.S. Pat. No. 4,670,620, have sought to increase selectivity in the dehydrating reaction of ethanol towards ethylene by using a zeolite at elevated temperatures. Typically diethyl ether is favored in the dehydration at lower reaction temperatures, but a mixture of ethylene and diethyl ether is still present. Currently, the dehydration may use solid acids, such as alumina, silica alumina, zeolites, tungstated or sulfated oxides, alumina phosphates, materials containing sulfonic acid functional groups, such as sulfonated polystyrene, sulfonated fluorocarbon polymers, sulfonic acid functionalized oxide materials (alumina, SBA-15, silica) and mixtures thereof.

The difficulties and expense in producing diethyl ether and ethanol are further related if the intended use of the diethyl ether is as a fuel additive or fuel for an engine. Compression ignition engines, e.g., diesel engines, are known to provide high fuel efficiencies. Typically, diesel engines operate with a fuel having a high cetane number and/or low boiling point. The combustion of diesel fuel, however, leads to many environmental concerns, e.g., environmentally unfriendly emissions. To reduce environmental pollution arising from combustion of diesel fuel, several attempts have been made in the past to replace and/or supplement diesel fuel with alternative fuels having reduced impact on the environment.

One well-known potential fuel source is ethanol, the combustion of which is much more environmentally-friendly than diesel fuel. Unfortunately, ethanol has a low cetane number and, as such, is not a suitable candidate for a diesel fuel. Additionally, the polarity, corrosivity, adhesiveness and friction properties of ethanol can complicate the use of ethanol in gasoline blends. It has been found, however, that when diethyl ether is combined with diethyl ether, the resultant ethanol/diethyl ether composition, unlike purified ethanol, performs well in compression engines. U.S. Publication No. 2008/0086933 shows that adding even 4 wt. % diethyl ether to an ethanol fuel increases the vapor pressure, thus improving fuel properties and engine performance. As such, the ethanol/diethyl ether composition provides an effective and environmentally-friendly alternative to traditional diesel fuel.

Unfortunately, diethyl ether presents many handling and storage problems. For example, diethyl ether has a high vapor pressure and presents a high risk of explosion when contacted with air. Thus, the need exists for a process for selectivity producing a composition comprising diethyl ether and/or a mixture of ethanol and diethyl ether that avoids the known problems associated with diethyl ether handling and storage.

### SUMMARY OF THE INVENTION

The present invention has found several novel catalysts for converting ethanol to diethyl ether under dehydrating conditions. These catalysts represent a significant improvement for producing diethyl ether and mixtures of diethyl ether and ethanol. Advantageously diethyl ether and mixtures thereof are produced with low ethylene formation.

In one embodiment of the present invention, the ethanol used as the starting material may be formed by hydrogenolysis of an ester selected from the group consisting of methyl acetate or ethyl acetate or hydrogenating acetic acid. Acetic acid may be formed by carbonylating methanol that is synthesized from hydrogen and carbon monoxide obtained by producing syngas. The syngas is obtained from coal, natural gas, petroleum or biomass.

Diethyl ether may be used as a fuel additive or as a fuel. In one embodiment, there is a process for producing a fuel additive comprising diethyl ether made by dehydrating ethanol using any of the catalysts disclosed herein. The fuel additive may further comprise ethanol, such as from 5 to 95 wt. % ethanol. In one aspect, the ethanol in the fuel additive is residual ethanol from the dehydrating or may be separately added to the fuel additive. The fuel additive comprising diethyl ether may be added to a fuel selected from the group consisting of gasoline, diesel, biofuel, and combinations thereof. In another embodiment, there is a process for producing a fuel comprising diethyl ether made by dehydrating ethanol using any of the catalysts disclosed herein. The fuel may further comprise ethanol that is either the residual ethanol from the dehydrating or separately added ethanol.

In a first aspect, the process for producing diethyl ether according to the present invention comprises dehydrating ethanol in the vapor phase in the presence of a catalyst to form diethyl ether, the catalyst comprising an alumina solid catalyst material and a metal impregnated thereon, wherein the metal is selected from the group consisting of tin, copper, platinum, palladium and mixtures thereof. The metal may be present in amount from 0.1 to 10 wt. %, preferably from 0.5 to 3 wt. %. This catalyst may have a conversion of the ethanol from 60 to 90%, and preferably at least 70%, and a selectivity to diethyl ether is at least 50%, preferably from 60 to 90%. This catalyst may also have selectivity to ethylene that is less than 10%, preferably less than 1%. The dehydrating process may be conducted at a pressure from 0 to 50 bar, e.g., from 0 to 40 bar, and a temperature from 150° C. to 450° C., e.g., from 250° C. to 350° C.

In a second aspect, the process for producing diethyl ether according to the present invention comprises dehydrating ethanol in the vapor phase in the presence of a catalyst to form diethyl ether, the catalyst comprising tin impregnated on a solid catalyst material selected from the group consisting of titania, zeolite, phosphotungstate and hydrotalcite. The zeolite may be Li-mordenite or Li-ZSM-5. The phophotungstate solid catalyst material may be monocesium tungstophosphate. The conversion of the ethanol is at least 70% with a selectivity to diethyl ether of at least 40%. The selectivity to ethylene is less than 10%. The dehydrating process may be conducted at a pressure from 0 to 50 bar, e.g., from 0 to 40 bar, and a temperature from 150° C. to 450° C., e.g., from 250° C. to 350° C.

In a third aspect, the process for producing diethyl ether according to the present invention comprises dehydrating ethanol in the vapor phase in the presence of a catalyst to form diethyl ether, the catalyst comprising a Group IA metal selected from the group consisting of lithium, sodium, cesium and potassium, e.g., preferably sodium, wherein the Group IA metal is impregnated on a silica support. The Group IA metal may be present from 0.1 to 40 wt. %, based on the total weight of the catalyst. The conversion of the ethanol is at least 70%, preferably at least 80%, with a selectivity to diethyl ether of at least 70%, e.g., at least 80%. The selectivity to ethylene is less than 10%. The dehydrating process may be conducted at a pressure from 0 to 50 bar, e.g., from 0 to 40 bar, and a temperature from 150° C. to 450° C., e.g., from 250° C. to 350° C.

In a fourth aspect, the process for producing diethyl ether according to the present invention comprises dehydrating ethanol in the vapor phase in the presence of a catalyst to form diethyl ether, the catalyst comprising a Group IA metal selected from the group consisting of lithium, sodium and potassium, e.g. preferably potassium, wherein the Group IA metal is impregnated on a magnesium oxide solid catalyst material. The Group IA metal may be present from 0.1 to 10 wt. %, e.g., from 0.1 to 5 wt. %, based on the total weight of the catalyst. The conversion of the ethanol is at least 70%, preferably at least 80%, with a selectivity to diethyl ether of at least 70%, e.g., at least 80%. The selectivity to ethylene is less than 10%. The dehydrating process may be conducted at a pressure from 0 to 50 bar, e.g., from 0 to 40 bar, and a temperature from 150° C. to 450° C., e.g., from 250° C. to 350° C.

In a fifth aspect, the process for producing diethyl ether according to the present invention comprises dehydrating ethanol in the vapor phase in the presence of a catalyst to form diethyl ether, the catalyst comprising copper impregnated on a hydroxyapatite solid catalyst material. Copper may be present from 0.1 to 10 wt. %, e.g., from 0.1 to 5 wt. %, based on the total weight of the catalyst. The conversion of the ethanol is at least 70%, preferably at least 80%, with a selectivity to diethyl ether of at least 70%, e.g., at least 80%. The selectivity to ethylene is less than 10%. The dehydrating process may be conducted at a pressure from 0 to 50 bar, e.g., from 0 to 40 bar, and a temperature from 150° C. to 450° C., e.g., from 250° C. to 350° C.

In a sixth aspect, the process for producing diethyl ether according to the present invention comprises dehydrating ethanol in the vapor phase in the presence of a catalyst to form diethyl ether, the catalyst comprising germanium impregnated on a solid catalyst material, wherein the solid catalyst material is selected from the group consisting of alumina and a zeolite, e.g., Li-ZSM-5. Germanium may be present from 0.1 to 10 wt. %. The catalyst may further comprise cesium. The molar ratio of germanium to cesium may be from 0.01:1 to 0.5:1. The conversion of the ethanol is at least 70%, preferably at least 80%, with a selectivity to diethyl ether of at least 70%, e.g., at least 80%. The selectivity to ethylene is less than 10%. The dehydrating process may be conducted at a pressure from 0 to 50 bar, e.g., from 0 to 40 bar, and a temperature from 150° C. to 450° C., e.g., from 250° C. to 350° C.

Specific catalysts within the scope of the present invention are provided below. Depending on the particular catalyst it may be more advantageous to operate at a particular pressure and temperature to achieve favorable conversions and selectivity.

In a first embodiment, the invention is directed to a process for producing diethyl ether, the process comprising dehydrating ethanol in the vapor phase at a pressure from 0 to 50 bar and a temperature from 150 to 450° C. in the presence of a catalyst to form diethyl ether, wherein the catalyst is selected from the group consisting of cesium and germanium impregnated on an alumina solid catalyst material; germanium impregnated on a Li-ZSM-5 solid catalyst material; copper impregnated on an alumina solid support material; palladium impregnated on an alumina solid catalyst material; platinum impregnated on an alumina solid catalyst material; tin impregnated on a monocesium tungstophosphate solid catalyst material; tin impregnated on a Li-ZSM-5 solid catalyst material; germanium impregnated on a Li-mordenite solid catalyst material; tin impregnated on a titania solid catalyst material; cesium impregnated on a silica support; tin impregnated on an Li-mordenite solid catalyst material; tin impregnated on an alumina solid catalyst material; sodium impregnated on a silica support; potassium impregnated on a magnesium oxide solid catalyst material; tin impregnated on a hydrotalcite solid catalyst material; and copper impregnated on a hydroxyapatite solid catalyst material.

In a second embodiment, the invention is directed to a process for producing diethyl ether, dehydrating ethanol in the vapor phase at a pressure from 25 to 40 bar and a temperature from 225° C. to 275° C. in the presence of a catalyst to form diethyl ether, the catalyst comprising cesium and germanium impregnated on an alumina solid catalyst material. Cesium may be present from 0.1 to 5 wt. % and wherein the germanium may be present from 0.1 to 5 wt. %. This catalyst may have a conversion of the ethanol that is at least 90% and a selectivity to diethyl ether that is at least 90%.

In a third embodiment, the invention is directed to a process for producing diethyl ether comprising dehydrating ethanol in the vapor phase at a pressure from 25 to 40 bar and a temperature from 225° C. to 275° C. in the presence of a catalyst to form diethyl ether, the catalyst comprising germanium impregnated on a Li-ZSM-5 solid catalyst material. Germanium may be present from 0.1 to 10 wt. %. This catalyst may have a conversion of the ethanol that is at least 65% and a selectivity to diethyl ether that is at least 90%.

In a fourth embodiment, the invention is directed to a process for producing diethyl ether comprising dehydrating ethanol in the vapor phase at a pressure from 10 to 20 bar and a temperature from 300° C. to 350° C. in the presence of a catalyst to form diethyl ether, the catalyst comprising copper impregnated on an alumina solid catalyst material. Copper may be present from 0.1 to 10 wt. %. This catalyst may have a conversion of the ethanol that is at least 75% and a selectivity to diethyl ether that is at least 60%.

In a fifth embodiment, the invention is directed to a process for producing diethyl ether comprising dehydrating ethanol in the vapor phase at a pressure from 5 to 15 bar and a temperature from 300° C. to 350° C. in the presence of a catalyst to form diethyl ether, the catalyst comprising palladium impregnated on an alumina solid catalyst material. Palladium may be present from 0.1 to 10 wt. %. This catalyst may have a conversion of the ethanol is at least 75% and a selectivity to diethyl ether is at least 70%.

In a sixth embodiment, the invention is directed to a process for producing diethyl ether comprising dehydrating ethanol in the vapor phase at a pressure from 10 to 20 bar and a temperature from 300° C. to 350° C. in the presence of a catalyst to form diethyl ether, the catalyst comprising platinum impregnated on an alumina solid catalyst material. Platinum may be present from 0.1 to 10 wt. %. This catalyst may have a conversion of the ethanol is at least 80% and a selectivity to diethyl ether is at least 55%.

In a seventh embodiment, the invention is directed to a process for producing diethyl ether comprising dehydrating ethanol in the vapor phase at a pressure from 0 to 10 bar and a temperature from 225° C. to 275° C. in the presence of a catalyst to form diethyl ether, the catalyst comprising tin impregnated on a monocesium tungstophosphate solid catalyst material. Tin may be present from 0.1 to 10 wt. %. This catalyst may have a conversion of the ethanol is at least 80% and a selectivity to diethyl ether is at least 40%.

In an eighth embodiment, the invention is directed to a process for producing diethyl ether comprising dehydrating ethanol in the vapor phase at a pressure from 10 to 20 bar and a temperature from 300° C. to 350° C. in the presence of a catalyst to form diethyl ether, the catalyst comprising tin impregnated on a monocesium tungstophosphate solid catalyst material. Tin may be present from 0.1 to 10 wt. %. This catalyst may have a conversion of the ethanol is at least 80% and a selectivity to diethyl ether is at least 45%.

In a ninth embodiment, the invention is directed to a process for producing diethyl ether comprising dehydrating ethanol in the vapor phase at a pressure from 10 to 20 bar and a temperature from 225° C. to 275° C. in the presence of a catalyst to form diethyl ether, the catalyst comprising tin impregnated on a Li-ZSM-5 solid catalyst material. Tin may be present from 0.1 to 10 wt. %. This catalyst may have a conversion of the ethanol is at least 80% and a selectivity to diethyl ether is at least 80%.

In a tenth embodiment, the invention is directed to a process for producing diethyl ether comprising dehydrating ethanol in the vapor phase at a pressure from 10 to 20 bar and a temperature from 300° C. to 350° C. in the presence of a catalyst to form diethyl ether, the catalyst comprising germanium impregnated on a Li-mordenite solid catalyst material. Germanium may be present from 0.1 to 10 wt. %. This catalyst may have a conversion of the ethanol is at least 80% and a selectivity to diethyl ether is at least 45%.

In an eleventh embodiment, the invention is directed to a process for producing diethyl ether comprising dehydrating ethanol in the vapor phase at a pressure from 5 to 15 bar and a temperature from 300° C. to 350° C. in the presence of a catalyst to form diethyl ether, the catalyst comprising tin impregnated on a titania solid catalyst material. Tin may be present from 0.1 to 10 wt. %. This catalyst may have a conversion of the ethanol is at least 75% and a selectivity to diethyl ether is at least 65%.

In a twelfth embodiment, the invention is directed to a process for producing diethyl ether comprising dehydrating ethanol in the vapor phase at a pressure from 30 to 40 bar and a temperature from 375° C. to 425° C. in the presence of a catalyst to form diethyl ether, the catalyst comprising cesium impregnated on a silica support. Cesium may be present from 1 to 5 wt. %. This catalyst may have a conversion of the ethanol is at least 90% and a selectivity to diethyl ether is at least 85%.

In a thirteenth embodiment, the invention is directed to a process for producing diethyl ether comprising dehydrating ethanol in the vapor phase at a pressure from 10 to 20 bar and a temperature from 225° C. to 275° C. in the presence of a catalyst to form diethyl ether, the catalyst comprising tin impregnated on an Li-mordenite solid catalyst material. Tin may be present from 0.1 to 10 wt. %. This catalyst may have a conversion of the ethanol is at least 80% and a selectivity to diethyl ether is at least 70%.

In a fourteenth embodiment, the invention is directed to a process for producing diethyl ether comprising dehydrating ethanol in the vapor phase at a pressure from 5 to 15 bar and a temperature from 300° C. to 350° C. in the presence of a catalyst to form diethyl ether, the catalyst comprising tin impregnated on an alumina solid catalyst material. Tin may be present from 0.1 to 10 wt. %. This catalyst may have a conversion of the ethanol is at least 75% and a selectivity to diethyl ether is at least 65%.

In a fifteenth embodiment, the invention is directed to a process for producing diethyl ether comprising dehydrating ethanol in the vapor phase at a pressure from 30 to 40 bar and a temperature from 225° C. to 275° C. in the presence of a catalyst to form diethyl ether, the catalyst comprising sodium impregnated on a silica support. Sodium may be present from 10 to 30 wt. %. This catalyst may have a conversion of the ethanol is at least 95% and a selectivity to diethyl ether is at least 95%.

In a sixteenth embodiment, the invention is a process for producing diethyl ether, the process comprising dehydrating ethanol in the vapor phase at a pressure from 30 to 40 bar and a temperature from 275° C. to 325° C. in the presence of a catalyst to form diethyl ether, the catalyst comprising potassium impregnated on a magnesium oxide solid catalyst material. Potassium is present from 0.1 to 10 wt. %. This catalyst may have a conversion of the ethanol is at least 95% and a selectivity to diethyl ether is at least 95%.

In a seventeenth embodiment, the invention is a process for producing diethyl ether, the process comprising dehydrating ethanol in the vapor phase at a pressure from 30 to 40 bar and a temperature from 225° C. to 275° C. in the presence of a catalyst to form diethyl ether, the catalyst comprising tin impregnated on a hydrotalcite solid catalyst material. Tin is present from 0.1 to 10 wt. %. This catalyst may have a conversion of the ethanol is at least 95% and a selectivity to diethyl ether is at least 95%.

In an eighteenth embodiment, the invention is a process for producing diethyl ether, the process comprising dehydrating ethanol in the vapor phase at a pressure from 30 to 40 bar and a temperature from 225° C. to 275° C. in the presence of a catalyst to form diethyl ether, the catalyst comprising copper impregnated on a hydroxyapatite solid catalyst material. Copper is present from 0.1 to 10 wt. %. This catalyst may have a conversion of the ethanol is at least 95% and a selectivity to diethyl ether is at least 95%.

### DETAILED DESCRIPTION OF THE INVENTION

I. Introduction

In general, the present invention relates to processes for producing diethyl ether by dehydration of ethanol in the presence of a dehydrating catalyst. The dehydration reaction may be in the vapor phase or may be in the liquid phase. Preferably, the dehydration occurs in the vapor phase. Due to the difficulties in storing diethyl ether, it is preferred to use a catalyst chosen to provide high conversion of ethanol and high selectivity to diethyl ether. This allows the diethyl ether to be prepared just prior to use since the process and catalyst are controlled to produce a known amount of diethyl ether. Additionally, the catalyst is chosen to have a low selectivity to by-products, particularly to have a low selectivity to ethylene. This reduces the requirement for further separation of the diethyl ether from undesirable by-products or impurities. The temperature and pressure of the reaction may also be adjusted to control ethanol conversion and selectivity of ethanol to diethyl ether.

The ethanol used to form the diethyl ether may be hydrous or anhydrous. In some embodiments, the ethanol may comprise less than 12 wt. % water, e.g., from 0.1 to 8 wt. % water or from 3 to 8 wt. % water. When hydrous ethanol is used, the water may be separated from the diethyl ether more easily than separating water from the ethanol. Without being bound by theory, the lower boiling point of diethyl ether as compared to ethanol decreases the difficulty in the water separation, particularly when ethanol conversion is high.

In some embodiments, the diethyl ether may be used as a fuel additive. In other embodiments, the diethyl ether may be used as a fuel. The fuel and fuel additive may each further comprise ethanol. The ethanol may be residual ethanol, e.g., unreacted ethanol, or the ethanol may be separately added to the fuel and fuel additive. The fuel additive may be added to a fuel selected from the group consisting of gasoline, diesel, biofuel, and combinations thereof. The fuel may then be used to power automotive engines, stationary engines and marine engines. In some embodiments, the fuel additive may be the fuel.

Generally, the dehydrating catalysts of the present invention comprise at least one active metal on a solid catalyst material. Without being bound by theory adding the active metal to the solid catalyst material is believed to improve the conversion of ethanol and selectivity to diethyl ether, with low formations of other compounds. The active metal may include, without limitation, sodium, potassium, tin, copper, germanium, palladium, platinum, cesium, lithium, and combinations thereof. The present invention has developed several different catalysts with a wide range of metal loadings. Typically the metal loadings on the dehydrating catalyst are lower for tin, copper, germanium, palladium, platinum, and cesium than for alkaline earth metals, such as sodium, potassium, and lithium. The active metal may be present in a loading of less than 50 wt. %. As described further below the metal loading will vary for

The dehydrating catalysts also include a solid catalyst material, also referred to herein as a support. The solid catalyst material may participate in the dehydration reaction and formation of the desired ether product. The solid catalyst materials may include inorganic metal oxides, such as silica, magnesia, titania, and alumina, zeolites, phosphotungstates, hydrotalcite and hydroxyapatite. The present invention has found particular combinations of active metals and solid catalyst material that achieve improved performance. In one embodiment the solid catalyst material is not functionalized with a sulfonated group or halide group. The morphology of the solid catalyst material may vary widely and includes pellets, extrudates, spheres, spray dried microspheres, rings, pentarings, trilobes, quadrilobes, multi-lobal shapes, or flakes, although cylindrical pellets are preferred.

As explained above, the inventive dehydrating catalysts are characterized by high conversion of ethanol and high selectivity to diethyl ether. For purposes of the present invention, the term “conversion” refers to the amount of ethanol in the feed that is converted to a compound other than ethanol. Conversion is expressed as a percentage based on acetic acid in the feed. The dehydrating catalysts of the present invention achieve conversion of ethanol that is at least 70%, e.g., at least 75%, at least 80%, at least 85%, at least 90%, at least 95% or 100%. Selectivity is expressed as a mole percent based on converted ethanol. It should be understood that each compound converted from ethanol has an independent selectivity and that selectivity is independent from conversion. The selectivity of ethanol to diethyl ether is at least 40%, e.g., at least 45%, at least 50%, at least 60%, at least 65%, at least 70%, at least 75%, at least 80%, at least 85%, at least 90%, at least 95% or 100%. The yield of diethyl ether may be calculated by multiplying the conversion of ethanol by the selectivity of ethanol to diethyl ether. In some embodiments, the yield is at least 30%, e.g., at least 40%, at least 50%, at least 60%, at least 70%, at least 80%, at least 90%, at least 95% or 100%. Selectivity of ethanol to ethylene is preferably suppressed by the dehydrating catalyst and process conditions, and is less than 10%, e.g., less than 7.5%, less than 5%, less than 3%, less than 1% or less than 0.1%. The dehydrating catalysts of the present invention do not require an alkyl halide to retard formation of ethylene.

II. Ethanol Dehydration Reaction

The ethanol may be vaporized at the reaction temperature, following which the vaporized ethanol may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of ethanol. In one embodiment, the ethanol may be vaporized at the boiling point of ethanol at the particular pressure, and then the vaporized ethanol may be further heated to the reactor inlet temperature. In another embodiment, the ethanol is mixed with other gases before vaporizing, followed by heating the mixed vapors up to the reactor inlet temperature.

The reactor, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed.

The dehydration in the reactor may be carried out in either the liquid phase or vapor phase. Preferably, the reaction is carried out in the vapor phase under the following conditions. The reaction temperature may range from 150° C. to 450° C., e.g., from 200° C. to 400° C. In some embodiments, the temperature ranges from 225° C. to 275° C. In other embodiments, the temperature ranges from 275° C. to 325° C. In yet other embodiments, the temperature ranges from 300° C. to 350° C. In still other embodiments, the temperature ranges from 375° C. to 425° C. The pressure may range from 0 to 50 bar, e.g., from 0 to 40 bar. In some embodiments, the pressure ranges from 0 to 10 bar. In other embodiments, the pressure ranges from 5 to 15 bar. In yet other embodiments, the pressure ranges from 10 to 20 bar. In still other embodiments, the pressures ranges from 30 to 40 bar. The reactants may be fed to the reactor at a gas hourly space velocity that ranges from 50 hr⁻¹ to 10,000 hr⁻¹, e.g., from 500 hr⁻¹ to 5,000 hr⁻¹, from 1000 hr⁻¹ to 5,000 hr⁻¹, or from 2500 hr⁻¹ to 4500 hr⁻¹. These reaction conditions may also vary depending on the dehydrating catalyst. Reaction conditions for the dehydration of ethanol with particular catalysts are described further herein.

Contact or residence time can also vary widely, depending upon such variables as amount of ethanol, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

III. Catalysts for Forming Diethyl Ether

As described above, the inventive catalysts have been found to have high conversion of ethanol and high selectivity to diethyl ether. The selectivity and conversion may be enhanced by tailoring the pressure and temperature of the dehydration reaction of ethanol.

a. Catalyst Comprising a Metal on an Alumina Solid Catalyst Material

In one embodiment, the inventive catalyst for dehydrating ethanol to form diethyl ether comprises a metal impregnated on an alumina solid catalyst material. Alumina solid catalyst materials may include gamma-alumina (γ-Al₂O₃), etu-alumina (η-Al₂O₃), kappa alumina (κ-Al₂O₃), theta-alumina (θ-Al₂O₃), or other alumina phase which is stable at temperatures use for catalyst calcination and conversion of primary alcohols, such as ethanol, to their corresponding olefin. Preferably, the alumina solid catalyst material is γ-alumina. The metal may be selected from the group consisting of tin, copper, platinum, palladium, and mixtures thereof. The metal may be present from 0.1 to 10 wt. %, e.g., from 0.1 to 5 wt. %, from 0.5 to 3 wt. %, or from 1 to 3 wt. %. Alumina may be present from 90 to 99.9 wt. %, e.g., from 95 to 99.9 wt. %, from 97 to 99.5 wt. %, or from 97 to 99 wt. %.

When using the metal-containing alumina catalyst to dehydrate ethanol in a reactor, the reactor may be operated at a temperature from 150° C. to 450° C., e.g., from 250° C. to 350° C. or from 300° C. to 350° C. The reactor may be operated at a pressure from 0 to 50 bar, e.g., from 0 to 40 bar, from 0 to 30 bar, or from 5 to 20 bar. To achieve higher conversions and selectivities the temperature and pressure may vary depending on the catalyst as described below. In general, the conversion of ethanol may be at least 75%, e.g., of at least 80% or at least 85%. In terms of ranges, conversion of ethanol may range from 75 to 98%, e.g., from 80 to 95% or from 80 to 90%. Without being bound by theory the addition of a metal on an alumina solid catalyst material may improve the conversion of ethanol, leading to potentially higher yields of diethyl ether. The selectivity of ethanol to diethyl ether may vary from at least 50%, e.g., of at least 65% or at least 70%. In terms of ranges, the ethanol may have a selectivity to diethyl ether from 50 to 75%. The ethanol preferably has a low selectivity to ethylene, e.g., less than 10%, less than 5% or less than 1%.

One specific example of the inventive catalyst described above is a catalyst comprising tin on alumina. The catalyst may comprise from 0.1 to 10 wt. % tin, e.g., from 0.1 to 5 wt. %, from 0.5 to 3 wt. %, or from 1 to 3 wt. %. To achieve conversions of at least 75% and selectivity to diethyl ether of at least 65%, the tin on alumina dehydration catalyst may be used in a reactor operating at a preferred temperature from 300° C. to 350° C. and a pressure from 5 to 15 bar, more preferably from 5 to 12 bar.

Another specific example of the inventive catalyst described above is a catalyst comprising copper on alumina. The catalyst may comprise from 0.1 to 10 wt. % copper, e.g., from 0.1 to 5 wt. %, from 0.5 to 3 wt. %, or from 1 to 3 wt. %. To achieve conversions of at least 75% and selectivity to diethyl ether of at least 65%, the copper on alumina dehydration catalyst may be used in a reactor operating at a preferred temperature from 300° C. to 350° C. and a pressure from 5 to 20 bar, more preferably from 12 to 20 bar.

Yet another specific example of the inventive catalyst described above is a catalyst comprising palladium on alumina. Without being bound by theory, the addition of palladium may improve selectivity to diethyl ether. The catalyst may comprise from 0.1 to 10 wt. % palladium, e.g., from 0.1 to 3 wt. %, from 0.1 to 2 wt. %, or from 0.2 to 1 wt. %. To achieve conversions of at least 75% and selectivity to diethyl ether of at least 70%, the palladium on alumina dehydration catalyst may be used in a reactor operating at a preferred temperature from 300° C. to 350° C. and a pressure from 5 to 20 bar, more preferably from 12 to 20 bar.

Another specific example of the inventive catalyst described above is a catalyst comprising platinum on alumina. The catalyst may comprise from 0.1 to 10 wt. % platinum, e.g., from 0.1 to 3 wt. %, from 0.1 to 2 wt. %, or from 0.2 to 1 wt. %. To achieve conversions of at least 75%, and more preferably a conversion of at least 85%, and selectivity to diethyl ether of at least 55%, the platinum on alumina dehydration catalyst may be used in a reactor operating at a preferred temperature from 300° C. to 350° C. and a pressure from 5 to 20 bar, more preferably from 12 to 20 bar.

b. Catalyst Comprising Tin on a Solid Catalyst Material

In one embodiment, the inventive catalyst for dehydrating ethanol to form diethyl ether comprises tin impregnated on a solid catalyst material selected from the group consisting of titania, zeolite, phosphotungstate and hydrotalcite (HT). Zeolite as used in the present application generally refers to microporous, aluminosilicate minerals. Examples of suitable zeolites include, but not limited to, silicoaluminophosphate (SAPO-34), clinoptilolite, Li-mordenite, ZSM-5, Li-ZSM-5, X-zeolite, and Y-zeolite. In other embodiments, the phosphotungstate solid catalyst material is monocesium tungstophosphate (CsH-PW₁₂). In one embodiment, HT may be natural occurring or synthetic, known as double layer hydroxides, including compounds having the structure [Mb₁₋ₓM2ₓ(OH)₂]^{x+}. [A_{x/m}^{m-}mH₂O]^{x-}, where M1 is Mg, Ca, Ni or Zn; M2 is Al, Fe, Mn, or Cr; A^{m-} is a anion such as carbonate, nitrate, or sulfate; m is greater than 1; x is between 0 and 1; and n is greater than x. An exemplary HT includes Mg₆Al₂CO₃(OH)₁₆.4(H₂O). The tin may be present from 0.1 to 10 wt. %, e.g., from 0.1 to 5 wt. %, from 0.5 to 3 wt. %, or from 1 to 3 wt. %. The solid catalyst material may be present from 90 to 99.9 wt. %, e.g., from 95 to 99.9 wt. %, from 97 to 99.5 wt. %, or from 97 to 99 wt. %.

When using the tin on solid catalyst material to dehydrate ethanol in a reactor, the reactor may be operated at a temperature from 150° C. to 450° C., e.g., from 200° C. to 400° C. or from 225° C. to 350° C. The reactor may be operated at a pressure from 0 to 50 bar, e.g., from 0 to 40 bar, from 0 to 30 bar, or from 0 to 20 bar. The conversion of ethanol may be at least 70%, e.g., at least 75%. In terms of ranges, conversion of ethanol may range from 70 to 100%, e.g., from 75 to 100%. The selectivity of ethanol to diethyl ether may be at least 40%. In terms of ranges, the ethanol may have a selectivity to diethyl ether from 40 to 100%. The ethanol preferably has a low selectivity to ethylene, e.g., less than 10%, less than 5% or less than 1%.

One specific example of the inventive catalyst described above is a catalyst comprising tin on HT. The catalyst may comprise from 0.1 to 10 wt. % tin, e.g., from 0.1 to 5 wt. %, from 0.5 to 3 wt. %, or from 1 to 3 wt. %. When using the inventive catalyst to dehydrate ethanol in a reactor, the reactor may be operated at a temperature from 150° C. to 450° C., e.g., from 200° C. to 300° C. or from 225° C. to 275° C. The reactor may be operated at a pressure from 0 to 50 bar, e.g., from 10 to 40 bar, or from 30 to 40 bar. Surprisingly and unexpectedly, the conversion of ethanol using a tin on HT dehydrating catalyst may be at least at least 95%, e.g., at least 99%, at least 99.5% or 100% and the selectivity of ethanol to diethyl ether may be at least 95%, e.g., at least 95%, or at least 97.5%.

Another specific example of the inventive catalyst described above is a catalyst comprising tin on phosphotungstate. The phosphotungstate may be monocesium tungstophosphate (CsH-PW₁₂). The catalyst may comprise from 0.1 to 10 wt. % tin, e.g., from 0.1 to 5 wt. %, from 0.5 to 3 wt. %, or from 1 to 3 wt. %. This catalyst is particular effective at lower temperatures and low pressures, and is also effective at higher temperatures and higher pressures. Thus, in one embodiment, when using the tin on phosphotungstate catalyst to dehydrate ethanol in a reactor, the reactor may be operated at a temperature 225° C. to 275° C. and at a pressure from 0 to 10 bar. The conversion of ethanol may be at least at least 80% and the selectivity of ethanol to diethyl ether may be at least 40%. In another embodiment, when using the tin on phosphotungstate catalyst to dehydrate ethanol in a reactor, the reactor may be operated at a temperature 300° C. to 350° C. and at a pressure from 10 to 20 bar. The conversion of ethanol may be at least at least 80% and the selectivity of ethanol to diethyl ether may be at least 40% or at least 45%.

Yet another specific example of the inventive catalyst described above is a catalyst comprising tin on zeolite. The catalyst may comprise from 0.1 to 10 wt. % tin, e.g., from 0.1 to 5 wt. %, from 0.5 to 3 wt. %, or from 1 to 3 wt. %. When using the tin on zeolite catalyst to dehydrate ethanol in a reactor, the reactor may be operated at a temperature from 150° C. to 450° C., e.g., from 200° C. to 300° C. or from 225° C. to 275° C. The reactor may be operated at a pressure from 0 to 50 bar, e.g., from 10 to 40 bar, or from 10 to 20 bar. When the zeolite solid catalyst material is Li-ZSM-5, the conversion of ethanol may be at least at least 80%, and the selectivity of ethanol to diethyl ether may be at least 80%. When the zeolite solid catalyst material is Li-mordenite, the conversion of ethanol may be at least at least 80%, and the selectivity of ethanol to diethyl ether may be at least 70%.

Another specific example of the inventive catalyst described above is a catalyst comprising tin on titania. The catalyst may comprise from 0.1 to 10 wt. % tin, e.g., from 0.1 to 5 wt. %, from 0.5 to 3 wt. %, or from 1 to 3 wt. %. When using the tin on titania catalyst to dehydrate ethanol in a reactor, the reactor may be operated at a temperature from 150° C. to 450° C., e.g., from 300° C. to 400° C. or from 300° C. to 350° C. The reactor may be operated at a pressure from 0 to 50 bar, e.g., from 0 to 20 bar, or from 0 to 10 bar. The conversion of ethanol may be at least at least 75% and the selectivity of ethanol to diethyl ether may be at least 65%.

c. Catalyst Comprising a Group IA Metal on a Silica Support

In one embodiment, the inventive catalyst for dehydrating ethanol to form diethyl ether comprises a Group IA metal impregnated on a silica support. The silica may have a surface area from 50 to 600 m²/g, e.g., from 100 to 500 m²/g or from 100 to 300 m²/g. For purposes of the present specification, surface area refers to BET nitrogen surface area, meaning the surface area as determined by ASTM D6556-04, the entirety of which is incorporated herein by reference. The silica support may have an average pore diameter from 5 to 100 nm, e.g., from 5 to 30 nm, from 5 to 25 nm or from 5 to 10 nm, as determined by mercury intrusion porosimetry, and an average pore volume from 0.5 to 2.0 cm³/g, e.g., from 0.7 to 1.5 cm³/g or from 0.8 to 1.3 cm³/g, as determined by mercury intrusion porosimetry.

The Group IA metal may be selected from lithium, sodium, cesium and potassium. In some embodiments, the Group IA metal is sodium. The Group IA metal may be present from 1 to 40 wt. %, e.g., from 2 to 30 wt. %, from 2.5 to 25 wt. %, or from 15 to 25 wt. %. The silica support may be present from 60 to 90 wt. %, e.g., from 65 to 90 wt. %, from 70 to 85 wt. %, or from 75 to 85 wt. %.

When using the inventive catalyst to dehydrate ethanol in a reactor, the reactor may be operated at a temperature from 150° C. to 450° C., e.g., from 200° C. to 400° C. or from 225° C. to 400° C. The reactor may be operated at a pressure from 0 to 50 bar, e.g., from 0 to 40 bar, from 20 to 40 bar, or from 30 to 40 bar. The conversion of ethanol may be at least 90%, e.g., at least 95%, at least 99%, at least 99.5% or 100%. In terms of ranges, conversion of ethanol may range from 90 to 100%, e.g., from 95 to 100%. The selectivity of ethanol to diethyl ether may be at least 85%, e.g., at least 90%, at least 95%, at least 99, at least 99.5% or 100%. In terms of ranges, the ethanol may have a selectivity to diethyl ether from 85 to 100%, e.g., from 90 to 100% from 95 to 100%. The ethanol preferably has a low selectivity to ethylene, e.g., less than 10%, less than 5% or less than 1%.

One specific example of the inventive catalyst described above is a catalyst comprising sodium on a silica support. The catalyst may comprise from 10 to 30 wt. % sodium, e.g., from 15 to 25 wt. %. When using the sodium on a silica support catalyst to dehydrate ethanol in a reactor, the reactor may be operated at a temperature from 150° C. to 450° C., e.g., from 200° C. to 300° C. or from 225° C. to 275° C. The reactor may be operated at a pressure from 0 to 50 bar, e.g., from 10 to 40 bar, or from 30 to 40 bar. The conversion of ethanol may be at least at least 95%, e.g., at least 99%, at least 99.5% or 100% and the selectivity of ethanol to diethyl ether may be at least 95%, e.g., at least 99%, or 100%.

Another specific example of the inventive catalyst described above is a catalyst comprising cesium on a silica support. The catalyst may comprise from 1 to 5 wt. % cesium, e.g., from 1 to 3 wt. %. When using the inventive catalyst to dehydrate ethanol in a reactor, the reactor may be operated at a temperature from 150° C. to 450° C., e.g., from 250° C. to 450° C. or from 375° C. to 425° C. The reactor may be operated at a pressure from 0 to 50 bar, e.g., from 10 to 40 bar, or from 30 to 40 bar. The conversion of ethanol may be at least at least 95%, e.g., at least 97.5%, or at least 99% and the selectivity of ethanol to diethyl ether may be at least 85%.

d. Catalyst Comprising a Group IA Metal on Magnesium Oxide

In one embodiment, the inventive catalyst for dehydrating ethanol to form diethyl ether comprises a Group IA metal impregnated on a magnesium oxide solid catalyst material. In this embodiment, the Group IA metal may preferably include lithium, sodium and potassium. In some embodiments, the Group IA metal is sodium. The Group IA metal may be present from 0.1 to 10 wt. %, e.g., from 0.1 to 5 wt. %, from 0.5 to 3 wt. %, or from 1 to 3 wt. %. Without being bound by theory lower loadings of Group IA metals may be used with magnesium oxide. The solid catalyst material may be present from 90 to 99.9 wt. %, e.g., from 95 to 99.9 wt. %, from 97 to 99.5 wt. %, or from 97 to 99 wt. %. In one embodiment, the Group IA metal may be impregnated on magnesium carbonate that forms magnesium oxide upon drying and calcination.

When using the inventive catalyst to dehydrate ethanol in a reactor, the reactor may be operated at a temperature from 150° C. to 450° C., e.g., from 200° C. to 350° C. or from 275° C. to 325° C. The reactor may be operated at a pressure from 0 to 50 bar, e.g., from 0 to 40 bar, from 20 to 40 bar, or from 30 to 40 bar. The conversion of ethanol may be at least 90%, e.g., at least 95%, at least 99%, at least 99.5% or 100%. In terms of ranges, conversion of ethanol may range from 90 to 100%, e.g., from 95 to 100%. The selectivity of ethanol to diethyl ether may be at least 90%, e.g., at least 95%, at least 99%, at least 99.5% or 100%. In terms of ranges, the ethanol may have a selectivity to diethyl ether from 90 to 100%, e.g., from 95 to 100%. The ethanol preferably has a low selectivity to ethylene, e.g., less than 10%, less than 5% or less than 1%.

One specific example of the inventive catalyst described above is a catalyst comprising potassium on magnesium oxide. The catalyst may comprise from 0.1 to 5 wt. % potassium. When using the inventive catalyst to dehydrate ethanol in a reactor, the reactor may be operated at a temperature from 150° C. to 450° C., e.g., from 250° C. to 350° C. or from 275° C. to 325° C. The reactor may be operated at a pressure from 0 to 50 bar, e.g., from 10 to 40 bar, or from 30 to 40 bar. The conversion of ethanol may be at least at least 95%, e.g., at least 99%, at least 99.5% or 100% and the selectivity of ethanol to diethyl ether may be at least 95%, e.g., at least 97.5%, or at least 99%.

e. Catalyst Comprising Copper on Hydroxyapatite

In one embodiment, the inventive catalyst for dehydrating ethanol to form diethyl ether comprises copper impregnated on a hydroxyapatite (HAP) solid catalyst material. The copper may be present from 0.1 to 10 wt. %, e.g., from 0.1 to 5 wt. %, from 0.5 to 5 wt. %, or from 1 to 5 wt. %. The HAP solid catalyst material may be present from 90 to 99.9 wt. %, e.g., from 95 to 99.9 wt. %, from 95 to 99.5 wt. %, or from 95 to 99 wt. %.

When using the inventive catalyst to dehydrate ethanol in a reactor, the reactor may be operated at a temperature from 150° C. to 450° C., e.g., from 200° C. to 350° C. or from 225° C. to 275° C. The reactor may be operated at a pressure from 0 to 50 bar, e.g., from 0 to 40 bar, from 20 to 40 bar, or from 30 to 40 bar. The conversion of ethanol may be at least 90%, e.g., at least 95%, at least 99%, at least 99.5% or 100%. In terms of ranges, conversion of ethanol may range from 90 to 100%, e.g., from 95 to 100%. The selectivity of ethanol to diethyl ether may be at least 90%, e.g., at least 95%. In terms of ranges, the ethanol may have a selectivity to diethyl ether from 90 to 100%. The ethanol preferably has a low selectivity to ethylene, e.g., less than 10%, less than 5% or less than 1%.

One specific example of the inventive catalyst described above is a catalyst comprising copper on HAP solid catalyst material. The catalyst may comprise from 0.1 to 5 wt. % potassium. When using the copper on HAP to dehydrate ethanol in a reactor, the reactor may be operated at a temperature from 225° C. to 275° C. The reactor may be operated at a pressure 30 to 40 bar. The conversion of ethanol may be at least at least 95% and the selectivity of ethanol to diethyl ether may be at least 90.

f. Catalyst Comprising Germanium

In one embodiment, the inventive catalyst for dehydrating ethanol to form diethyl ether comprises germanium impregnated on a solid catalyst material. The solid catalyst material may be selected from the group consisting of alumina and a zeolite. Suitable alumina and zeolite solid catalyst materials are described above. Preferably, the zeolite is Li-ZSM-5 or Li-mordenite. The germanium may be present from 0.1 to 10 wt. %, e.g., from 0.1 to 5 wt. %, from 0.5 to 5 wt. %, or from 1 to 5 wt. %. In some embodiments, germanium may be co-impregnated with another metal such as cesium. The solid catalyst material may be present from 90 to 99.9 wt. %, e.g., from 95 to 99.9 wt. %, from 95 to 99.5 wt. %, or from 95 to 99 wt. %.

When using the inventive catalyst to dehydrate ethanol in a reactor, the reactor may be operated at a temperature from 150° C. to 450° C., e.g., from 200° C. to 400° C. or from 225° C. to 375° C. The reactor may be operated at a pressure from 0 to 50 bar, e.g., from 0 to 40 bar, from 10 to 40 bar, or from 15 to 40 bar. The conversion of ethanol may be at least 70%, e.g., at least 80%, at least 90, at least 95%, at least 99%, at least 99.5% or 100%. In terms of ranges, conversion of ethanol may range from 70 to 100%, e.g., from 80 to 100% or from 90 to 100%. The selectivity of ethanol to diethyl ether may be at least 65%, e.g., at least 70% or at least 80%. In terms of ranges, the ethanol may have a selectivity to diethyl ether from 65 to 100%, e.g., from 70 to 100%. The ethanol preferably has a low selectivity to ethylene, e.g., less than 10%, less than 5% or less than 1%.

One specific example of the inventive catalyst described above is a catalyst comprising germanium and cesium on an alumina solid catalyst material. The catalyst may comprise from 0.1 to 5 wt. % germanium, e.g., from 0.5 to 3 wt. %, or from 1 to 3 wt. %. The catalyst may also comprise from 0.1 to 5 wt. % cesium, e.g., from 0.5 to 3 wt. %, or from 1 to 3 wt. %. When using the inventive catalyst to dehydrate ethanol in a reactor, the reactor may be operated at a temperature from 225° C. to 275° C. The reactor may be operated at a pressure 30 to 40 bar. This catalyst may have a conversion of ethanol of at least 95%, e.g., of at least 97.5%, at least 99%, at least 99.5% or 100% and a selectivity of ethanol to diethyl ether of at least 95%, e.g., of at least 97.5%.

Another specific example of the inventive catalyst described above is a catalyst comprising germanium on Li-ZSM-5. The catalyst may also comprise from 0.1 to 5 wt. % germanium, e.g., from 0.5 to 3 wt. %, or from 1 to 3 wt. %. When using the inventive catalyst to dehydrate ethanol in a reactor, the reactor may be operated at a temperature from 225° C. to 275° C. The reactor may be operated at a pressure 30 to 40 bar. This catalyst may have a conversion of ethanol of at least 70% and a selectivity of ethanol to diethyl ether of at least 95%.

Yet another specific example of the inventive catalyst described above is a catalyst comprising germanium on Li-mordenite. The catalyst may also comprise from 0.1 to 5 wt. % germanium, e.g., from 0.5 to 3 wt. %, or from 1 to 3 wt. %. When using the inventive catalyst to dehydrate ethanol in a reactor, the reactor may be operated at a temperature from 375° C. to 425° C. The reactor may be operated at a pressure 10 to 20 bar. This catalyst may have a conversion of ethanol of at least 80% and a selectivity of ethanol to diethyl ether of at least 65%.

g. Catalyst Production

The present invention also relates to processes for making the catalyst. In one embodiment, the solid catalyst material, or a precursor to the solid catalyst material, is impregnated with a precursor to the metals describe above. When multiple metals are impregnated there may be multiple precursor solutions that are impregnated in sequential or simultaneously steps. With simultaneous impregnation, it may be desired to employ a dispersion agent, surfactant, or solubilizing agent, e.g., ammonium oxalate or an acid such as acetic or nitric acid, to facilitate the dispersing or solubilizing of the metal precursors in the event the precursors are incompatible with the desired solvent, e.g., water. The use of a solvent, such as water, glacial acetic acid, a strong acid such as hydrochloric acid, nitric acid, or sulfuric acid, or an organic solvent, is preferred for impregnating a metal onto the solid catalyst material. The solution is stirred and combined with the solid catalyst material using, for example, incipient wetness techniques in which the metal precursor is added to a solid catalyst material having the same pore volume as the volume of the solution. Impregnation occurs by adding, optionally drop wise, a solution containing the metal precursors to the dry solid catalyst material. Capillary action then draws the metal into the pores of the solid catalyst material. After impregnation, the solution is dried and calcined. Drying may occur, for example, at a temperature from 50° C. to 300° C. for a period from 1 to 24 hours. Following drying, the solution is calcined with ramped heating, for example, at a temperature from 300° C. to 900° C. for a period of time from 1 to 12 hours to form the final catalyst. Upon heating and/or the application of vacuum, the metal(s) of the precursor(s) preferably decompose into their oxide or elemental form. In some cases, the completion of removal of the solvent may not take place until the catalyst is placed into use and/or calcined, e.g., subjected to the high temperatures encountered during operation. During the calcination step, or at least during the initial phase of use of the catalyst, such compounds are converted into a catalytically active form of the metal or a catalytically active oxide thereof.

Suitable metal precursors may include, for example, metal halides, amine solubilized metal hydroxides, metal nitrates or metal oxalates. For example, suitable compounds for platinum precursors and palladium precursors include chloroplatinic acid, ammonium chloroplatinate, amine solubilized platinum hydroxide, platinum nitrate, platinum tetra ammonium nitrate, platinum chloride, platinum oxalate, palladium nitrate, palladium tetra ammonium nitrate, palladium chloride, palladium oxalate, sodium palladium chloride, sodium platinum chloride, and platinum ammonium nitrate, Pt(NH₃)₄(NO₄)₂.

After the drying and calcining of the catalyst is completed, the catalyst may be reduced in order to activate it. Reduction is carried out in the presence of a reducing gas, preferably hydrogen. The reducing gas is optionally continuously passed over the catalyst at an initial ambient temperature that is increased up to 400° C. In one embodiment, the reduction is carried out after the catalyst has been loaded into the reaction vessel where the hydrogenation will be carried out.

IV. Diethyl Ether Products and Uses

As a solvent, diethyl ether is polar and may be used in liquid-liquid extractions due to its limited solubility in water. Diethyl ether may also be used in reactions involving organometallic reagents. For this applications, catalysts described herein that achieve higher conversions and selectivity may be preferred to reduce the necessary purification of diethyl ether.

As explained above, diethyl ether may be used for a variety of purposes, including as a solvent, as a fuel additive and as a fuel. In particular using bio fuels, such as bio ethanol, present challenges when incorporating into different types of engines. Bio ethanol, due to water concentration of lower grades, is often unable to burn satisfactory in traditional engines. In particular, the high water content of bio ethanol limits its use for transportation and requires an energy-consuming distillation stage to remove the excess water. Furthermore, bio ethanol often suffer the disadvantage of low cetane numbers, and they are not suitable for diesel engine operation unless expensive additives are added. Diethyl ether may be used as a fuel additive and in particular may be obtained from bio ethanol. Diethyl ether has a high cetane number, ranging from 85-96, and finds frequent use as a starting fluid for gasoline and diesel engines. Diethyl ether is highly volatile and has a low flash point. Due to its high cetane number, diethyl ether may be added to existing ethanol-fuel blends to increase the cetane number. The ethanol-fuel blend may be used in a gasoline or diesel engine for automobile, stationary and marine purposes.

Diethyl ether may be added to an ethanol fuel additive for use in a compression engine. Current ethanol-diesel blends may comprise 10, 15, 20 or 30 wt. % ethanol, commonly referred to as E10, E15, E20 and E30 ethanol-diesel blends. As compared to pure diesel fuel, ethanol-diesel blends may have decreased smoke emissions while maintaining similar engine performance. However, each 10 wt. % addition of ethanol to diesel reduces the cetane number by 7.1. This cetane number reduction may result in increased ignition delay, reduced combustion duration, high maximum pressure rates, and slightly decreased gas temperature, as explained by Lei et al., “Performance and Emission Characteristics of Diesel Engine Fueled with Ethanol-Diesel Blends in Different Altitude Regions,” Journal of Biomedicine and Biotechnology, vol. 11, p. 1-10.

To combat this cetane reduction, diethyl ether may be added to the ethanol-diesel blend. In some embodiments, diethyl ether may be added from 1 to 75 wt. % diethyl ether, or from 25-75 wt. %, based on the total amount of ethanol in the ethanol-diesel blend.

Additionally, diethyl ether may be used as a fuel, alone or in combination with ethanol. In some embodiments, ethanol may be the main fuel component, comprising at least 40 wt. % or at least 50 wt. % of the fuel, with diethyl ether present from 1 to 50 wt. %, e.g., from 4 to 40 wt. % or from 4 to 20 wt. %. In other embodiments, diethyl ether may be the main fuel component, with diethyl ether present from 45 to 95 wt. %, e.g., from 50 to 90 wt. % or from 55 to 85 wt. % The fuel may also comprise water. The amount of water may depend on whether hydrous or anhydrous ethanol is used to prepare the diethyl ether. Water content in the ethanol and diethyl ether fuel may range from 0 to 30 wt. %.

Depending on the desired end use of the diethyl formed using the inventive catalysts described above, the diethyl ether product may be further processed. The diethyl ether product may be dried to remove water or other impurities such as ethylene. Additionally, depending on the ethanol conversion, ethanol can be separated from the diethyl ether product, retained, or added separately. For example, if the ethanol dehydration reaction has a higher conversion of near 100% and higher selectivity to diethyl ether of near 100%, no further treatment would be necessary if the diethyl ether was to be used as a solvent. However, if the diethyl ether is intended to be used in an ethanol-diethyl ether fuel additive or fuel blend, ethanol would be added to the diethyl ether. If the ethanol dehydration reaction has 70% conversion with 95% selectivity to diethyl ether, the diethyl ether would have 30% residual ethanol and be ready for use as a diethyl ether-ethanol fuel additive or diethyl ether-ethanol fuel blend. The inventive catalysts allow for the diethyl ether product to be controlled to the desired conversion and selectivity to produce a product ready for the desired end use.

V. Ethanol Sources

In various embodiments, the production of diethyl ether may be integrated with upstream processes to produce ethanol, and reactants used to produce ethanol. In one embodiment, the production of diethyl ether may be integrated with a process that converts an ester, such as methyl acetate, ethyl acetate, or mixtures thereof, to ethanol, which is then further dehydrated in the presence of the one of the catalyst described herein to diethyl ether. In one embodiment, the production of diethyl ether may be integrated with a process that converts acetic acid to ethanol by hydrogenation, which is then further dehydrating in the presence of the one of the catalyst described herein to diethyl ether.

The ethanol used to form the diethyl ether may be obtained from a variety of sources, including from ethylene hydrogenation, bio-fermentation, acetic acid hydrogenation, ester hydrogenolysis and/or wood pyrolysis. Ethylene hydrogenation is described in U.S. Pat. No. 3,686,334, the entirety of which is hereby incorporated by reference.

The ester used in ester hydrogenolysis may comprise methyl acetate or ethyl acetate. For example, U.S. Pat. No. 8,088,832 discloses a method and apparatus for synthesizing ethanol using stepwise catalytic reaction to convert carbon monoxide and hydrogen into ethanol through intermediates, such as methanol and methyl acetate, using catalysts including iridium acetate. U.S. Pat. No. 8,080,693 discloses a process for converting methanol to ethanol which comprises reacting methanol and carbon monoxide in the presence of a catalyst to produce a product comprising at least 25 mole % methyl acetate and, in some instances, acetic acid. U.S. Pat. No. 4,454,358 discloses a process for continuously producing ethanol via the carbonylation of methanol and hydrogenating a mixture of methanol and methyl acetate to form ethanol. Additional hydrogenolysis catalysts include copper containing catalysts. These copper containing catalysts may further comprise one or more additional metals, optionally, on a catalyst support. The optional additional metal or metals may be selected from Group IB, IIB, IIIB, IVB, VB, VIB, VIIB, VIII transition metals, a lanthanide metal, an actinide metal or a metal selected from any of Groups IIIA, IVA, VA, and VIA. Particular metal combinations for some exemplary catalyst compositions include copper/cobalt/zinc, copper/zinc/iron, copper/cobalt/zinc/iron, copper/cobalt/zinc/iron/calcium, and copper/cobalt/zinc/molybdenum/sodium. Particular copper containing catalysts may comprise copper chromite, copper and zinc, and/or copper-zinc-oxide. Exemplary catalysts are further described in U.S. Pat. Nos. 5,198,592; 5,414,161; and 7,947,746; U.S. Pub. No. 2009/0326080, and WO83/03409, the entireties of which are incorporated herein by reference. Hydrogenolysis catalysts may comprise CuO or ZnO. However, CuO and ZnO may be reduced or partially reduced by hydrogen during the course of the hydrogenolysis reaction. It is also possible to pre-reduce CuO and/or ZnO by passing hydrogen over the catalyst before the introduction of the methyl acetate feed.

Similarly, ethyl acetate may be used in ester hydrogenolysis to prepare ethanol, as described in copending U.S. application Ser. No. 13/299,730, the entire contents and disclosures of which are hereby incorporated by reference. The hydrogenolysis reactor may comprise any suitable type of reactor, such as a fixed bed reactor or a fluidized bed reactor. The hydrogenolysis process may be operated in a vapor phase, or a mixed vapor/liquid phase regime. The hydrogenolysis reaction may change from a mixed vapor/liquid phase to a fully vapor phase reaction, as the reaction proceeds down the reactor. The reaction may occur in the presence of a copper-based catalyst. Copper-based catalyst may comprise copper chromite, copper and zinc, and/or copper-zinc-oxide. Other copper-based catalyst may include an MgO—SiO₂ support that is impregnated with copper. In other embodiments, the catalyst may be a Group VIII-based catalyst comprising a Group VIII metal selected from the group consisting of iron, ruthenium, osmium, cobalt, rhodium, iridium, nickel, palladium, and platinum. In addition, there may be one or more secondary promoter metals selected from the group consisting of zinc, cobalt, tin, germanium, lead, rhenium, tungsten, molybdenum. Group VIII-based catalysts may advantageously be supported on any suitable support known to those skilled in the art; non-limiting examples of such supports include carbon, silica, titania, clays, aluminas, zinc oxide, zirconia and mixed oxides.

Another route to form ethanol is to hydrogenate acetic acid. The hydrogenation reaction may be represented as follows:

HOAc+2H₂→EtOH+H₂O

The raw materials, acetic acid and hydrogen, fed to the reactor used in connection with the process of this invention may be derived from any suitable source including natural gas, petroleum, coal, biomass, and so forth. As examples, acetic acid may be produced via methanol carbonylation, acetaldehyde oxidation, ethane oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from other carbon sources have drawn increasing interest. In particular, when petroleum is relatively expensive, it may become advantageous to produce acetic acid from synthesis gas (“syngas”) that is derived from other available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. The microorganism employed in the fermentation process may be Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteroides amylophilus and Bacteroides ruminicola. Optionally, in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. No. 6,509,180, and U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. Black liquor, which is an aqueous solution of lignin residues, hemicellulose, and inorganic chemicals, may also be used as a biomass source. Biomass-derived syngas has a detectable ¹⁴C isotope content as compared to fossil fuels such as coal or natural gas.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into syngas, and U.S. Pat. No. 6,685,754, which discloses a method for the production of a hydrogen-containing gas composition, such as a syngas including hydrogen and carbon monoxide, are incorporated herein by reference in their entireties.

The acetic acid fed to the hydrogenation reactor may also comprise other carboxylic acids and anhydrides, as well as aldehyde and/or ketones, such as acetaldehyde and acetone. Preferably, the feed stream comprises acetic acid and ethyl acetate. A suitable acetic acid feed stream comprises one or more of the compounds selected from the group consisting of acetic acid, acetic anhydride, acetaldehyde, ethyl acetate, diethyl acetal, diethyl ether, and mixtures thereof. These other compounds may also be hydrogenated in the processes of the present invention. In some embodiments, the presence of carboxylic acids, such as propanoic acid or its aldehyde, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the hydrogenation reactor without the need for condensing the acetic acid and light ends or removing water, saving overall processing costs.

The following examples describe the procedures used for the preparation of various catalysts employed in the process of this invention.

VI. Integration of Diethyl Production with Ethanol Production

The production of diethyl ether may be integrated with the production of ethanol. As described herein, ethanol may be produced by acetic acid hydrogenation. Therefore, in one embodiment, diethyl ether formation may be integrated with acetic acid hydrogenation. In one exemplary embodiment, the diethyl ether production and ethanol production may occur in one reaction zone with a hydrogenation catalyst to hydrogenate acetic acid to ethanol and with a dehydration catalyst to dehydrate ethanol to diethyl ether. The catalysts may be present in layered catalyst beds. The hydrogenation catalyst may be chosen to have high conversion of acetic acid and high selectivity to ethanol.

In another embodiment, diethyl ether formation may be integrated with acetic acid hydrogenation and with acetic acid formation. As described herein, acetic acid may be formed by carbonylating methanol and/or a derivative thereof in the presence of a catalyst.

In yet another embodiment, diethyl ether formation may be integrated with ethanol formation via acetic acid hydrogenation, with acetic acid formation via carbonylation, and with methanol synthesis via syngas.

In still another embodiment, diethyl ether formation may be integrated with ethanol formation via acetic acid hydrogenation, with acetic acid formation via carbonylation, methanol synthesis via syngas, and with the production of syngas obtained from coal, natural gas, petroleum or biomass.

Integrating the diethyl ether process with acetic acid hydrogenation, methanol carbonylation, methanol synthesis and/or the production of syngas allows the processes to have increased energy and cost efficiencies.

The following examples describe the procedures used for the preparation of various catalysts employed in the process of this invention.

### EXAMPLES

### Example A

### 18 Wt. % Sodium on Silica

SiO₂ (extrudate) was crushed to a particle size of 0.85 mm and 1.18 mm. 8.1 g of NaNO₃ was dissolved in 8 g of H2O, followed by impregnating to the above SiO₂ support by stepwise incipient wetness using the rotating dryer. The obtained sample was dried in an oven at 120° C. for 5 hours, followed by calcination using the following temperature program: start at 60° C., ramp to 300° C. at 2° C./min, hold at 300° C. for 5 hours, followed by cooling to room temperature.

### Example B

### 3 wt. % Sn-HT

0.355 g of SnO and 10 g of hydrotalcite (Mg₆Al₂CO₃(OH)₁₆.4(H₂O)) were mixed together via mortar and pestle (3 wt. % Sn). These were mixed for approximately 5 minutes until the black and white powders appeared homogenous. The mixture was then calcined using the following temperature program: start at 60° C., ramp to 600° C. at 5° C./min, hold at 600° C. for 5 hours, followed by cooling to room temperature. The Sn containing Mg-μl HT powder was then pressed at 18,150 kg for 1 hour to form pellets, followed by lightly crushing the above pellets to a particle size of 0.85 mm and 1.18 mm for further use.

### Example C

### 3 wt. % K—MgO

10 g of magnesium carbonate was measured and pressed at 18,150 kg for 1 hour to form pellets. The pellets were then lightly crushed to a particle size of 0.85 mm and 1.18 mm for future use. 0.78 g of potassium acetate was dissolved in 10 g of acetone and a few drops of ethanol (about 0.5 ml) was added to allow uniform colloidal dispersion. The resulting clear colloid was impregnated to the pre-shaped MgCO₃ by stepwise incipient wetness using a rotating dryer. The catalyst was slow dried overnight in air (50° C./ramp 0.5° C./min). The catalyst was then dried for 8 hours in air at 120° C. (ramp 2° C./min). The catalyst was then calcined in air at 425° C. for 4 hours (ramp 0.5° C./min), followed by cooling to room temperature.

### Example D

### 4.3 wt. % Ge-Cs-γ-Al₂O₃

The catalyst was synthesized using a sequential impregnation method with Ge on the inner layer and Cs on the outer layer. The γ-Al₂O₃ (extrudate) was crushed to a particle size of 0.85 mm and 1.18 mm. To prepare the first layer, 1.01 g of bis(2-carboxyethyl) germanium (IV) sesquioxide was added to 25 g of distilled water and heated at about 65° C. until the solution was clear, followed by adding 5 g of acetone. The resulted clear solution was impregnated to 5 g of γ-Al₂O₃ by stepwise incipient wetness using a rotating dryer. The obtained sample was dried in an oven at 120° C. for 5 hours, followed by calcination using the following temperature program: start at 60° C., ramp to 500° C. at 2° C./min, hold at 500° C. for 5 hours, followed by cooling to room temperature. To prepare the second layer: 9.7 g of CsNO₃ was dissolved in 10 g of H₂O. The resulting solution was slowly added to 5 g of above Ge treated γ-Al₂O₃ in the reactor by stepwise incipient wetness using the rotating dryer. The obtained samples were dried in an oven at 120° C. for 5 hours, followed by calcination using the following temperature program: start at 60° C., ramp to 500° C. at 2° C./min, hold at 500° C. for 5 hours, followed by cooling to room temperature.

### Example E

### 1 wt. % Cu-HAP

The hydroxyapatite (HAP) powder was pressed at 18,150 kg for 1 hour to form pellets, followed by lightly crushing the pellets to a particle size of 0.85 mm and 1.18 mm for further use. 0.356 g of Cu(NO₃)₂.2H₂O was dissolved in 5 g of H₂O, followed by impregnating to HAP by stepwise incipient wetness using the rotating dryer. The obtained sample was dried in an oven at 120° C. for 8 hours, followed by calcination using the following temperature program: start at 60° C., ramp to 500° C. at 2° C./min, hold at 500° C. for 5 hours, followed by cooling to room temperature.

### Example F

### 3 wt. % Ge—Li-ZSM-5

Li-ZSM-5 (extrudate) was crushed to a particle size of 0.85 mm and 1.18 mm. 0.36 g of bis(2-carboxyethyl) germanium (IV) sesquioxide was added to 25 g of distilled water and heated at about 65° C. until the solution was clear, followed by adding 1 g of acetone. The resulting clear solution was impregnated to 5 g of Li-ZSM-5 by stepwise incipient wetness using a rotating dryer. The obtained samples were dried in an oven at 120° C. for 5 hours, followed by calcination using the following temperature program: start at 60° C., ramp to 500° C. at 2° C./min, hold at 500° C. for 5 hours, followed by cooling to room temperature

### Example G

### 3 wt. % Sn-γAl₂O₃

The γ-Al₂O₃ (extrudate) was crushed to a particle size of 0.85 mm and 1.18 mm. 10 g of the crushed γ-Al₂O₃ were added to a round bottom reactor. 0.3 g tin oxalate and 0.3 g ammonium oxalate were dissolved in 5 g H₂O. The solution was then impregnated onto the crushed γ-Al₂O₃ by stepwise incipient wetness technique using a rotating dryer. The obtained samples were dried in an oven at 120° C. for 5 hours, followed by calcination using the following temperature program: start at 60° C., ramp to 500° C. at 2° C./min, hold at 500° C. for 5 hours, followed by cooling to room temperature.

### Example H

### 3 wt. % Cu-γ-Al₂O₃

Example H was prepared using the same method as Example G, except that 1.41 g Cu(NO₃)₂.2H₂O was dissolved in 5 g H₂O. Additionally, the temperature program differed as follows: start at 60° C., ramp to 300° C. at 2° C./min, hold at 300° C. for 5 hours, followed by cooling to room temperature.

### Example I

### 3 wt. % Pd-γ-Al₂O₃

Example I was prepared using the same method as Example G, except that 0.77 g of Pd(NO₃)₂.2H₂O was dissolved in 5 g H₂O.

### Example J

### 3 wt. % Pt-γ-Al₂O₃

Example J was prepared using the same method as Example G, except that 1.41 g 0.61 g of Pt(NH₃)₄(NO₃)₂ was dissolved in 5 g H₂O.

### Examples K and L

### 3 wt. % Sn-CsH-PW₁₂

0.54 g of tin oxalate was dissolved in 0.5 g ammonium oxalate and 5 g of water, followed by impregnating to CsH-PW₁₂O₄₀ by stepwise incipient wetness using a rotating dryer. The obtained sample was dried in an oven at 120° C. for 8 hours, followed by calcination using the following temperature program: start at 60° C., ramp to 500° C. at 2° C./min, hold at 500° C. for 5 hours, followed by cooling to room temperature

### Example M

### 3 wt. % Sn—Li-Mordenite

Li-Mordenite (extrudate) was crushed to a particle size of 0.85 mm and 1.18 mm: 0.56 g of ammonium oxalate was dissolved in 10 g of distilled H₂O, followed by adding 0.54 g of Tin oxalate to the above solution. The resulting mixture was then heated at 60° C. until the solution turned clear, and then 1 g of acetone was added to the solution. The resulting clear solution was impregnated to 10 g of Li-Mordenite by stepwise incipient wetness using the rotating dryer. The obtained samples were dried in an oven at 120° C. for 5 hours, followed by calcination using the following temperature program: start at 60° C., ramp to 500° C. at 2° C./min, hold at 500° C. for 5 hours, followed by cooling to room temperature.

### Example N

### 3 wt. % Ge—Li-Mordenite

Example N was prepared using the same method as Example M, except that 0.72 g of bis(2-carboxyethyl) germanium (IV) sesquioxide was dispersed in 6 g of distilled H₂O. 1.1 g of oxalic acid hydrate was dissolved in 2 g of distilled water, and then slowly added to the above Ge suspension. The resulted mixture was then heated at 50° C. until the solution turned clear; followed by impregnating to the Li-Mordenite by stepwise incipient wetness using a rotating dryer.

### Example O

### 3 wt. % Sn—Li-ZSM-5

Li-ZSM-5 (extrudate) was crushed to a particle size of 0.85 mm and 1.18 mm. 0.56 g of ammonium oxalate was dissolved in 10 g of distilled H₂O, followed by adding 0.54 g of tin oxalate to the above solution. The resulting mixture was then heated at 60° C. until the solution turned clear, and then 1 g of acetone was added to the above solution. The resulted clear solution was impregnated to 10 g of Li-ZSM-5 by stepwise incipient wetness using the rotating dryer. The sample was dried in an oven at 120° C. for 5 hours, followed by calcination using the following temperature program: start at 60° C., ramp to 500° C. at 2° C./min, hold at 500° C. for 5 hours, followed by cooling to room temperature.

### Example P

### 3 wt. % Sn—TiO₂

The TiO₂ (extrudate) was crushed to a particle size of 0.85 mm and 1.18 mm. 5 g of pre-shaped SiO2 was measured and placed in a rotating flask. 0.3 g tin oxalate and 0.3 g ammonium oxalate were dissolved in 5 g H₂O. The solution was then impregnated onto, followed by impregnating to the TiO₂ by stepwise incipient wetness using the rotating dryer. The sample was dried in an oven at 120° C. for 5 hours, followed by calcination using the following temperature program: start at 60° C., ramp to 500° C. at 2° C./min, hold at 500° C. for 5 hours, followed by cooling to room temperature.

### Example O

### 2.5 wt. % Cs—SiO₂

The SiO₂ (extrudate) was crushed to a particle size of 0.85 mm and 1.18 mm for the following experiments: 5 g of pre-shaped SiO₂ was measured and placed in a rotating flask; 0.199 g of CsNO3 was dissolved in 20 g of H₂O, followed by impregnating to the SiO₂ by stepwise incipient wetness using the rotating dryer. The sample was dried in an oven at 120° C. for 5 hours, followed by calcination using the following temperature program: start at 60° C., ramp to 500° C. at 2° C./min, hold at 500° C. for 5 hours, followed by cooling to room temperature.

### Evaluation of Catalyst Properties

A fixed bed gas flow catalytic reactor was used as a reactor. 3 ml of above prepared catalysts was filled in a stainless steel tube reactor with a diameter of 9.5 mm″. As a pretreatment, hydrogen reduction was conducted for 1 hour under a carrier gas atmosphere (10% H₂/N₂ base; flow rate 125 ml/min) at 400° C. After the pretreatment, the testing was conducted at a temperature range of 250° C. to 400° C. and a pressure range of 0 to 51 bar, nitrogen flow rate at 125 sccm and ethanol flow rate at 0.2 ml/min. The reaction duration ranged from 5 to 80 hrs.

TABLE 1
Inventive Catalysts Ethanol Conversion, Selectivity to Diethyl Ether and Yield

| | | Ethanol | Diethyl Ether | | Process |
|---|---|---|---|---|---|
| Example | Catalyst | Conversion % | Selectivity % | Yield (%) | Conditions |
| A | 18 wt. % Na—SiO₂ | 100 | 100 | 100 | 250° C. |
| | | | | | 34 bar |
| B | 3 wt. % Sn-HT | 100 | 99 | 99 | 250° C. |
| | | | | | 34 bar |
| C | 3 wt. % K—MgO | 100 | 99 | 99 | 290° C. |
| | | | | | 34 bar |
| D | 4.3 wt. % Cs/Ge-γ-Al₂O₃ | 100 | 98 | 98 | 250° C. |
| | | | | | 34 bar |
| E | 1 wt. % Cu-HAP | 100 | 95 | 95 | 250° C. |
| | | | | | 34 bar |
| F | 3 wt. % Ge—Li-ZSM-5 | 70 | 97 | 68 | 250° C. |
| | | | | | 34 bar |
| G | 3 wt. % Sn-γ-Al₂O₃ | 79 | 68 | 54 | 325° C. |
| | | | | | 10 bar |
| H | 3 wt. % Cu-γ-Al₂O₃ | 81 | 65 | 53 | 325° C. |
| | | | | | 17 bar |
| I | 3 wt. % Pd-γ-Al₂O₃ | 78 | 72 | 56 | 325° C. |
| | | | | | 10 bar |
| J | 3 wt. % Pt-γ-Al₂O₃ | 86 | 56 | 48 | 325° C. |
| | | | | | 17 bar |
| K | 3 wt. % Sn—CsH-PW₁₂ | 83 | 41 | 34 | 250° C. |
| | | | | | 0 bar |
| L | 3 wt. % Sn—CsH-PW₁₂ | 84 | 48 | 32 | 325° C. |
| | | | | | 17 bar |
| M | 3 wt. % Sn—Li-Mordenite | 83 | 73 | 61 | 250° C. |
| | | | | | 17 bar |
| N | 3 wt. % Ge—Li-Mordenite | 84 | 67 | 56 | 325° C. |
| | | | | | 17 bar |
| O | 3 wt. % Sn—Li-ZSM-5 | 82 | 82 | 67 | 250° C. |
| | | | | | 17 bar |
| P | 3 wt. % Sn—TiO₂ | 79 | 68 | 54 | 325° C. |
| | | | | | 10 bar |
| Q | 2.5 wt. % Cs—SiO₂ | 99 | 88 | 87 | 400° C. |
| | | | | | 34 bar |

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing diethyl ether comprising:
dehydrating ethanol in the vapor phase at a pressure and a temperature in the presence of a catalyst to form diethyl ether, the catalyst comprising tin impregnated on a solid catalyst material selected from the group consisting of titania, zeolite, phosphotungstate and hydrotalcite.

2. The process of claim 1, wherein the catalyst is tin impregnated on hydrotalcite, the pressure is from 30 to 40 bar and the temperature is from 225° C. to 275° C.

3. The process of claim 1, wherein the catalyst is tin impregnated on a monocesium tungstophosphate solid catalyst material, the pressure is from 0 to 10 bar and the temperature is from 225° C. to 275° C.

4. The process of claim 1, wherein the catalyst is tin impregnated on a monocesium tungstophosphate solid catalyst material, the pressure is from 10 to 20 bar and the temperature is from 300° C. to 350° C.

5. The process of claim 1, wherein the catalyst is tin impregnated on a Li-ZSM-5 solid catalyst material, the pressure is from 10 to 20 bar and the temperature is from 225° C. to 275° C.

6. The process of claim 1, wherein the catalyst is tin impregnated on titania, the pressure is from 5 to 15 bar and the temperature is from 300° C. to 350° C.

7. The process of claim 1, wherein the catalyst is tin impregnated on an Li-mordenite solid catalyst material, the pressure is from 10 to 20 bar and the temperature is from 225° C. to 275° C.

8. A process for producing a fuel additive comprising diethyl ether made by the process of claim 1.

9. A process for producing diethyl ether comprising:
dehydrating ethanol in the vapor phase at a pressure and a temperature in the presence of a catalyst to form diethyl ether, wherein the catalyst is selected from the group consisting of:
(i) germanium impregnated on a solid catalyst material, wherein the solid catalyst material is selected from the group consisting of alumina and a zeolite;
(ii) a Group IA metal selected from the group consisting of lithium, sodium, cesium and potassium, wherein the Group IA metal is impregnated on a silica support;
(iii) a Group IA metal selected from the group consisting of lithium, sodium and potassium, wherein the Group IA metal is impregnated on a magnesium oxide solid catalyst material; and
(vi) copper impregnated on a hydroxyapatite solid catalyst material.

10. The process of claim 9, wherein the catalyst further comprises cesium and germanium impregnated on alumina, the pressure is from 25 to 40 bar and the temperature is from 225° C. to 275° C.

11. The process of claim 9, wherein the catalyst is germanium impregnated on a Li-ZSM-5 solid catalyst material, the pressure is from 25 to 40 bar and the temperature is from 225° C. to 275° C.

12. The process of claim 9, wherein the catalyst is germanium impregnated on a Li-mordenite solid catalyst material, the pressure from 10 to 20 bar and the temperature from 300° C. to 350° C.

13. The process of claim 9, wherein the catalyst is cesium impregnated on a silica support, the pressure is from 30 to 40 bar and the temperature is from 375° C. to 425° C.

14. The process of claim 9, wherein the catalyst is sodium impregnated on a silica support, the pressure is from 30 to 40 bar and the temperature is from 225° C. to 275° C.

15. The process of claim 9, wherein the catalyst is potassium impregnated on a magnesium oxide solid catalyst material, the pressure is from 30 to 40 bar and the temperature is from 275° C. to 325° C.

16. The process of claim 9, wherein the catalyst is copper impregnated on a hydroxyapatite solid catalyst material, the pressure is from 30 to 40 bar and the temperature is from 225° C. to 275° C.

17. A process for producing a fuel additive comprising diethyl ether made by the process of claim 1.

18. A process for producing diethyl ether comprising:
dehydrating ethanol in the vapor phase at a pressure and a temperature in the presence of a catalyst to form diethyl ether, the catalyst comprising an alumina solid catalyst material and a metal impregnated thereon, wherein the metal is selected from the group consisting of tin, copper, platinum, palladium and mixtures thereof.

19. The process of claim 10, wherein the catalyst comprises palladium, the pressure is from 5 to 15 bar and the temperature is from 300° C. to 350° C.

20. A process for producing a fuel additive comprising diethyl ether made by the process of claim 18.
